(19) United States
(12) Patent Application Publication
(10) Pub. No.: US 2010/0036467 A1
(43) Pub. Date: Feb. 11, 2010

(54) STIMULATION DEVICE FOR OSTEOSYNTHESIS AND ENDOPROSTHETICS

(75) Inventors: Werner Kraus, Munchen (DE); Stephanie Kraus, Bad Tolz (DE); Heribert Stephan, Munchen (DE)

Correspondence Address: DOBRUSIN & THENNISCH PC, 29 W LAWRENCE ST, SUITE 210, PONTIAC, MI, 48342, US

(73) Assignee: Neue Magnetodyn GmbH, Munchen (DE)

(21) Appl. No.: 12/298,398

(22) PCT Filed: Apr. 27, 2007
(86) PCT No.: PCT/DE2007/000764

§ 371 Date: Jun. 1, 2009

(30) Foreign Application Priority Data
May 1, 2006 (DE) 10 2006 019 955.3
Jul. 17, 2006 (DE) 10 2006 032 957.0

(51) Int. Cl. A61N 1/05

(52) U.S. Cl. 607/116

(57) ABSTRACT

The invention relates to a stimulation device for implanting in a human body, comprising a coil arrangement, a first electrode that is connection to a first pole of the coil arrangement and a second electrode that is connected to a second pole of the coil arrangement. According to the invention, the second electrode is configured as an elastic contact element.

The invention relates to a stimulation device for the implantation into the human body comprising a coil arrangement, a first electrode connected to a first pole of the coil arrangement and a second electrode connected to a second pole of the coil arrangement.

Such stimulation devices are known in the fields of osteosynthesis as well as endoprosthetics.

Osteosynthesis serves the strain-stable fixation of the fragments of a broken or ill bone in its uninjured, natural form by implanted screws, support plates, wires, bone marrow nails and the like, which are, in general, manufactured of stainless steel or titanium alloys. These osteosynthesis means enable the rapid mobilisation of the patient in combination with the simultaneous immobilisation of the damaged bone, which is an essential prerequisite for its recovery.

Endoprosthetics serves the implantation of prostheses, particularly joint prostheses, for example in the hip.

The number of patients having bone- and joint-bearing as well as supporting metal implants in the skeleton has increased exponentially in the past two decades. The reasons for this are the increase in complicated traumatic bone fractures and particularly the degenerative diseases of the joints (arthroses, necroses) which lead to an artificial joint replacement by an endoprosthesis in an increasingly earlier age. With the increase of the average age of people by almost ten years—during the past five decades—the claim for the trouble-free life of an artificial joint is also growing. If in the sixth to seventh decade of the past century this was fulfilled with 15 to 20 years, the technology is now confronted with the problem of ensuring a to the largest possible extent lossless mobility of the bearer of an artificial joint for up to three decades or more. There are efforts to meet these increasing requirements relating to the biomechanical tolerance of the biological bearing of a permanent implant in the skeleton with more compatible materials such as titanium alloys and patient-specific designs in combination with the maximum possible preservation of the sustaining vessels.

Despite of the remarkable progresses in the adjustment of the foreign body implants to the individual biological and physiological conditions, new problems arise in connection with the increasing requirements of the patients with respect to the mobility and life of the implant which require a stimulating mediation between the foreign body and its biological bearing. That this object can, even in cases of the extreme bone reparation failure, be solved by the application of extremely low-frequency alternating electromagnetic fields having a frequency of 3 to 30 Hz with a pure sinusoidal form (harmonic part <1%) in connection with an implanted coil (secondary inductivity of the so-called transformer) electrically connected to the metal components of the osteosynthesis and the joint endoprosthetics was proved and published in numerous basic experiments and clinical studies by the applicant within three and a half decades. The majority of the patients having supporting or joint implants were infected with germs which are nowadays referred to as biologically multi-resistant (MRSA=multi-resistant staphylococcus aureus) and which pose an increasing problem in the orthopedic and trauma surgery clinic. Apparently germs settling on permanent implants in the form of “bio films” and protecting themselves by mucous jackets are no longer accessible to antibiotics. The adherence of germ films on metal implants can apparently be prevented by the electric activation of their surface by the electromagnetic induction according to the method.

The technique of the transmittance functions according to the principle of the transformer: The injured or ill body region is flooded by an extremely low-frequency sinusoidal magnetic field having a frequency of approximately 1 to 100 Hz—preferably of 3 to 30 Hz—and a magnetic flux density of 0.5 to 5 mT (5 to 50 Gauss) generated by a functional current generator in one or more—primary—outer current coils into which the body part provided with the osteosynthesis means or the endoprosthesis is inserted. These extremely low-frequency electromagnetic fields permeate the tissue including possible clothing and a plaster cast as well as the non-magnetic (austenitic) support metals of the osteosynthesis or the endoprosthetics to a large extent without loss. A—secondary—coil arrangement, the so-called transformer, is implanted in an electric contact with these. The electro-potentials induced in the transformer will thus become effective in the area of the bony lesion as well as generally in the tissue adjacent to the osteosynthesis means or the endoprosthesis. The electric voltage, the frequency, the intensity, the signal form and the duration of the treatment determined by the indication-specific programming of the functional current generator determining the induced magnetic field serve as treatment parameters.

Basically therefore techniques for reducing the risks of osteosynthesis as well as endoprosthetics are available.

What is problematic, however, is, in particular, the situation in which an endoprosthesis or osteosynthesis means have been implanted for an extended period of time without the qualification to apply the therapy utilising the described electromagnetic alternating fields and an exchange of the supporting or joint implant in the cure-resistant infected bone poses a risk no longer calculable to the surgeon. Particularly for many, most of the time older patients with supporting and joint implants at risk of infection the complicated operation for exchanging an implant is accompanied by a significantly increased risk of life.

The invention is based on the object to provide a technology for avoiding the necessity of an implant exchange, particularly in case of high-risk patients.

Said object is solved by the features of the independent claim.

Advantageous embodiments of the invention are specified in the dependent claims.

The invention is based on the generic stimulation device in that the second electrode is formed as an elastic contact element. In this way it becomes possible to electrically connect metal parts implanted in the bone section via the elastic contact element. In this way the metal part already implanted will become an electrode while the part of the stimulation device electrically connected to the coil arrangement will form the associated counter electrode. Correspondingly the implant can be included in the therapy described in the introduction without being exchanged, using low-frequency electromagnetic alternating fields.

Usefully it is contemplated that the stimulation device comprises a shaft defining an axis, the coil arrangement is disposed in a radially inner accommodation area of the shaft, and at least a part of the shaft forms the first electrode. The stimulation device is therefore formed as an elongated element whereby it is suitable for an insertion into small orifices of the body and particularly the bone. The coil arrangement may be safely accommodated inside of the shaft of the stimulation device in a liquid- and gas-tight manner.

The invention is advantageously further developed in that an electrically insulating end piece through which an electric connection to the elastic contact element arranged at the side of the end piece opposing the shaft is lead is attached to an end section of the shaft. The electrically insulating end piece serves to insulate the elastic contact element from the remainder of the electrically conductive device body, and it further enables the realisation of the electric connection of the coil arrangement arranged in the shaft to the contact element disposed on the outside.

It may be contemplated that the contact element is fixed in the end piece. For example, the contact element may be sintered in or tipped in by means of epoxy resin; additional fixation means are therefore not required.

According to a variant of the present invention it is contemplated that the contact element, at least partly, consists of spring-hard steel.

It may also be contemplated that the contact element at least partly consists of spring-hard titanium.

For establishing a good electric contact between the contact element and the already implanted metal part it is usefully contemplated that the contact element comprises at least one undulated wire.

The invention may also be designed so that the contact element comprises at least one helical wire.

The stimulation device is preferably formed as a bone screw comprising a male thread. A bone screw can be advantageously deployed since it can be securely fixed in bone so that the relative position of the stimulation device with respect to the already implanted metal part will also not or only insignificantly change. Furthermore no other appliance has to be implanted to fix the bone screw. Even if the design of the stimulation device as a bone screw may be preferred, it is to be understood that all other forms are feasible. Sometimes the implantation of additional fixation means is required to fix stimulation devices of another form.

Above that the invention is further developed in a particularly useful way in that the outer surface of the stimulation device is at least partly provided with an electrically conductive coating enlarging the surface of the stimulation device and preventing the deposit of bacteria. Bactericidal coatings are known. If an electrically conductive bactericidal coating enlarging the surface of the stimulation device is selected, an enhancement of the bactericidal effect is achieved, namely due to the enlarged surface for the transmission of the electric field to the surrounding tissue.

In this connection it is preferable that the coating contains silver. A silver coating may, for example, be directly applied to implants of steel or titanium alloys by means of a sputtering technique.

Usefully, however, it may also be contemplated that a porous intermediate layer is provided between the surface of the device and the coating. The electrically conductive connection between the coating and the surface disposed under the intermediate layer of the stimulation device is provided by the surrounding body fluid and/or by a direct contact between the silver particles and the surface. The porous intermediate layer consists, for example, of ceramics or a plastic material.

The invention is based on the finding that a permanent conductive contact can be established between a stimulation device, particularly a bone screw, comprising an integrated secondary induction coil and a tongue-shaped electrode at the tip of the device and the surface of a metallic support or joint implant by means of a minimally invasive surgical procedure. With the induction of the secondary coil by means of an external electromagnetic field the surface of the permanent implant will become an electrode having an electric potential difference of 500 to 700 mV relative to the shaft of the stimulation device. With this arrangement particularly the following effects are achieved:
1. The deposition of germs is prevented.
2. The multi-resistance against antibiotics is eliminated.
3. The bone will grow towards the permanent implant and will render it firmly set again.

The invention will now be explained by way of example on the basis of preferred embodiments with reference to the accompanying drawings in which:

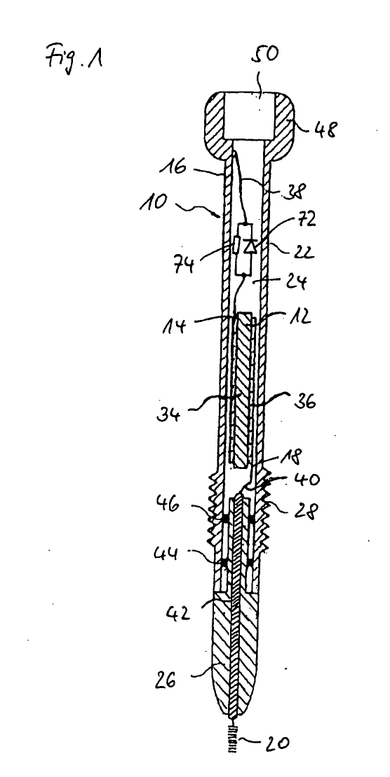

FIG. 1 shows a cross sectional view of a stimulation device according to the invention;

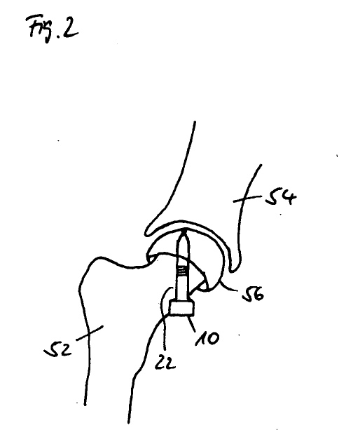

FIG. 2 shows a schematic illustration of a stimulation device introduced into a thigh bone for establishing a contact to a femoral head cap prosthesis;

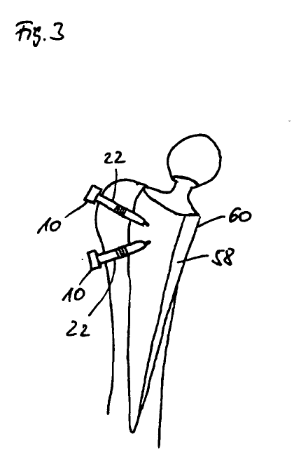

FIG. 3 shows a schematic illustration of two stimulation devices for establishing a contact to the shaft of a hip prosthesis screwed into the thigh bone;

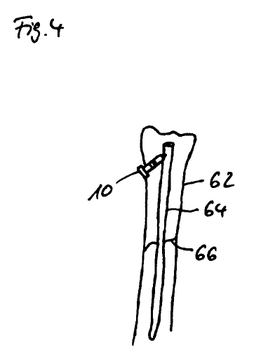

FIG. 4 shows a schematic illustration of a stimulation device for establishing a connection to a marrow nail introduced into a tubular bone;

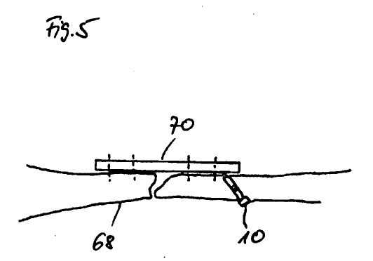

FIG. 5 shows a schematic illustration of a stimulation device for establishing a contact to a support plate introduced into a broken bone; and

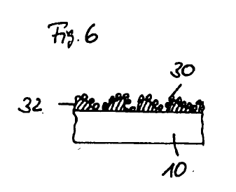

FIG. 6 shows a cross sectional view through the surface of a stimulation device according to the invention comprising a coating enlarging the surface.

In the following description of the preferred embodiments of the present invention the same numerals designate the same or comparable components.

FIG. 1 shows a cross sectional view of a stimulation device according to the invention for establishing a contact to a femoral head cap. The stimulation device is formed as a bone screw 10 having a male thread 28. The male thread 28 is provided in the distal area of the bone screw 10. Depending on the application it may also be useful to provide the male thread in the proximal area of the bone screw. In an accommodation area 24 surrounded by the shaft 22 of the bone screw 10 a coil arrangement 12 is provided. The coil arrangement 12 comprises a magnetic core 34 and a winding 36 attached thereto. A first pole 14 of the coil arrangement 12 is connected to the electrically conductive shaft 22 of the bone screw 14 forming the first electrode 16 via an electric connection 38 and a rectifier arrangement 72, 74. The rectifier arrangement comprises a diode 72 and an ohmic resistance 74 connected in parallel to the diode 72. The second pole 18 of the coil arrangement 12 is connected to an elastic contact element 20 disposed on the distal end of the bone screw 10 and forming the second electrode via another electric connection 40. For this purpose the electric connection 40 is lead through an electrically insulating end piece 26 consisting, for example, of a ceramic material or polyethylene. To this end the end piece 26 is provided with a central bore 42. Seals 44, 46 are provided to ensure that the accommodation area 24 of the coil arrangement 12 is closed towards the outer region of the bone screw 10 in a gas- and liquid-tight manner. Any other measures for a gas- and liquid-tight insertion of the end piece 26 into the shaft 22 of the bone screw 10 are also feasible. The bone screw 10 comprises a screw head 48 comprising an orifice 50 for inserting a turning tool at its proximal end. The orifice 50 may, for example, form an internal hexagon. The rectifier circuit realised by the diode 72 may have an advantageous effect on the localisation of the bone formation. In this way the first electrode 16 will form an anode at which the osteogenesis is suppressed or even an osteolysis will take place while the contact element 20 and the implant contacted by it (see, for example, FIG. 2) will form a cathode so that the bone formation is advanced particularly in the vicinity of the implant. With ohmic resistance 74 connected in parallel to the diode 72, an incomplete rectification is provided. When the mentioned advantages of the rectification are abandoned the rectifier arrangement 72, 74 is dispensable with so that the first pole 14 of the coil arrangement 12 can be directly connected to the first electrode 16.

FIG. 2 shows a schematic illustration of a stimulation device introduced into in a thigh bone. A thigh bone 52 and a pelvic bone 54 are shown. A femoral head cap prosthesis 56 is provided on the thigh bone 52. Such a femoral head cap prosthesis is frequently the origin and source of bacterial cultures spreading below the femoral head cap prosthesis 56. By contacting the femoral head cap prosthesis 56 via the bone screw 10—the distal section of the bone screw 10 actually hidden by the femoral head cap prosthesis 56 is also shown—the femoral head cap prosthesis 56 will form an electrode while the shaft 22 of the bone screw 10 forms the counter electrode. Consequently the tissue present between the electrodes is stimulated when external magnetic fields are applied.

FIG. 3 shows a schematic illustration of two stimulation devices for establishing a contact to the shaft of a hip prosthesis screwed into the thigh bone. In the present case the shaft 58 of a hip prosthesis 60 is contacted by two bone screws 10 of the type according to the invention and in this way forms the common counter electrode to the respective shafts 22 of the bone screws 10.

FIG. 4 shows a schematic illustration of a stimulation device for establishing a contact to a marrow nail introduced into a tubular bone. A tubular bone 62 including a fracture 66 stabilised by a marrow nail 64 is shown. The marrow nail 64 becomes an electrode due to a bone screw 10 according to the invention screwed into the tubular bone 62.

FIG. 5 shows a schematic illustration of a broken bone stabilised by a support plate. The broken bone 68 is stabilised by a metal plate 70. The screw joints of the metal plate 70 are indicated by broken lines. With a bone screw 10 according to the invention being screwed into the bone 68 and contacting the metal plate 70, the latter will also become an electrode.

FIG. 7 shows a cross sectional view through the surface of a stimulation device according to the invention. The outer surface of the stimulation device 10 is provided with an electrically conductive coating enlarging the surface and preventing the deposit of bacteria, preferably of silver particles 30 present in a colloidal state. The coating of the surface is mediated by a porous intermediate layer 32 which, for example, consists of a plastic or ceramic material. It is also possible that the silver particles are additionally or alternatively embedded in the intermediate layer. This can be realised by applying a ceramic-silver emulsion. The electric contact between the surface of the stimulation device 10 and the electrically conductive coating 30 is provided by body fluid or by a direct contact between the surface of the stimulation device 10 and the coating 30 in the area of the pores of the porous surface. Owing to the bactericidal coating the deposit of bacteria is constricted even without electric potentials provided via the surface of the stimulation device. Within the framework of the present invention this effect is amplified by the induced electric fields. Further also the effect of the induced electric field on the surrounding tissue is promoted since the electrically conductive coating enlarges the contact surface between the tissue and the electrode. As a result the positive biological effects can be enhanced in this way, or simpler and smaller devices can be used while maintaining a given quality, which, in particular, relates to the coil arrangement and the devices generating the external magnetic alternating field.

The features of the invention disclosed in the above description, in the drawings as well as in the claims may be important for the realisation of the invention individually or in any combination.

### LIST OF NUMERALS

10 bone screw
12 coil arrangement
14 first pole
16 electrode
18 second pole
20 contact element
22 shaft
24 accommodation area
26 end piece
28 male thread
30 coating
32 intermediate layer
34 magnetic core
36 winding
38 electric connection
40 electric connection
42 bore
44 sealing
46 sealing
48 screw head
50 orifice
52 thigh bone
54 pelvic bone
56 femoral head cap prosthesis
58 shaft
60 hip prosthesis
62 tubular bone
64 marrow nail
66 fracture
68 broken bone
70 metal plate
72 diode
74 ohmic resistance

1. A stimulation device for the implantation into the human body comprising a coil arrangement, a first electrode connected to a first pole of the coil arrangement and a second electrode connected to a second pole of the coil arrangement, characterised in that the second electrode is formed as an elastic contact element by which an implant can be contacted in its implanted position.

2. The stimulation device according to claim 1, characterised in that
the stimulation device comprises a shaft defining an axis,
the coil arrangement is arranged in a radially inner accommodation area of the shaft, and
at least a part of the shaft forms the first electrode.

3. The stimulation device according to claim 2, characterised in that an electrically insulating end piece through which an electric connection to the elastic contact element disposed on the side of the end piece opposing the shaft is lead is attached to an end section of the shaft.
